(19) United States
(12) Patent Application Publication
(10) Pub. No.: US 2014/0005572 A1
(43) Pub. Date: Jan. 2, 2014

(54) GUIDING DEVICE FOR URINE COLLECTION

(71) Applicant: Kuo-Huang Yang, Taipei (TW)

(72) Inventor: Kuo-Huang Yang, Taipei (TW)

(21) Appl. No.: 14/005,384

(22) PCT Filed: Mar. 12, 2012
(86) PCT No.: PCT/CN12/00305

§ 371 Date: Sep. 16, 2013

(30) Foreign Application Priority Data
Mar. 16, 2011 (CN) 20110062390.1

(51) Int. Cl. A61B 10/00

(52) U.S. Cl. CPC A61B 10/007
USPC 600/580

(57) ABSTRACT

Disclosed is a guiding device for urine collection (10) comprising a bottom portion (11), a body (12), a cup opening (14) and a first liquid discharge outlet (15). The body (12) is arranged so as to extend around the bottom portion (11), the bottom portion (11) and part of the body (12) thus forming a collection portion (13); the cup opening (14) is located on a side of the body (12) opposite to the bottom portion (11), and the first liquid discharge outlet (15) is arranged on the body (12) between the cup opening (14) and the collection portion (13). The guiding device for urine collection (10) relieves users from having consciously to control urine flow during urine collection and, at the same time, avoids contamination of hands, clothing, or surroundings in case of overflow due to excessive urine.

### BACKGROUND

1. Technical Field

The disclosure relates to a guiding device for urine collection.

2. Description of Related Art

The inspection personnel usually provide a paper cup and a test tube when people need to do an urine test. People must rely on physiological mechanism to control the flow of urine so as to save the urine in the paper cup, and then pour to the test tube from the paper cup.

However, the practice is inconvenient rely on physiological mechanisms to control the flow of urine to be saved in a paper cup. Users are hard to control urine, and easy to make urine overflow and dirty hands, clothing or the environment, particularly the elderly, children, pregnant women or patients with urinary system diseases will be more difficult to control.

In addition, due to the physical structure of the relationship of the female user. When the paper cup close to the body is difficult to judge the adequacy of the amount of urine, and when the paper cup away from the body, it is not easy to control urine flows into the paper cup.

Furthermore, it is an other test of vision, stability and coordination to pour the urine into the test tube.

Therefore, how to provide an urination device, it is possible to make the user in the state without deliberately control the flow of urine, and urine can be discharged to the default destination is one of important subjects.

### SUMMARY OF THE INVENTION

In view of the foregoing, the invention is to provide a guiding device for urine collection to make the user can discharge the urine into the default destination without deliberately control the flow of urine to overcome the deficiencies of the prior art.

A guiding device for urine collection comprises a bottom portion, a body, a cup opening and a first liquid discharge outlet. The body is arranged to extend around the bottom portion. The bottom portion and a part of the body form a collection portion. The cup opening is located on one side of the body opposite to the bottom portion. The first liquid discharge outlet is arranged on the body between the cup opening and the collection portion.

In one embodiment, a profile of one side of the cup opening is a curved surface to fit body curve, so that the cup opening can be more closely contact body.

In one embodiment, the guiding device for urine collection further comprises a wing located on the body adjacent to the cup opening. The wing allows the user to be able to hold the wing to operate the guiding device for urine collection. In addition, an adhesive layer may be provided on the wing, which is distant from the side of the cup opening (the side for contacting fingers), so that the fingers can be securely affixed to hold the wing.

In one embodiment, the body has a concave portion extended to the bottom portion. The collection portion is located between the bottom portion and an extension line of the top edge of the concave portion. The first liquid discharge outlet is located at a top edge of the concave portion.

In one embodiment, the overlooking profile of the cup opening is circular, oval, teardrop-shaped, egg-shaped, rectangular, rhombic, or triangular.

In one embodiment, one end of the overlooking profile of the cup opening is a sharp surface or v-shaped, that is used to meet the open angle of the two fingers, thus the fingers and the guiding device for urine collection are more closely so that the guiding device for urine collection is more easily operation.

In one embodiment, the guiding device for urine collection further comprises a second liquid discharge outlet located at the body of the collection portion. In another embodiment the second liquid discharge outlet is located at the bottom portion.

In one embodiment, the guiding device for urine collection further comprises a bag connected to the body. The first liquid discharge outlet is covered by the bag. In another embodiment, the first liquid discharge outlet and the second liquid discharge outlet are covered by the bag.

In one embodiment, the bag is a zipper bag, which can be sealed to prevent the urine leakage after collecting the urine.

The beneficial effects of the present invention is that a collection portion is formed in the lower body part, and the first liquid discharge outlet in the top of the collection portion, so it can allow users to prevent the amount of the urine is too many to overflow, and dirty hands, clothing or the environment in the collecting process. In addition, the urine stored in the collection portion can be poured into the test tube through the first liquid discharge outlet, without the user to control how much of the amount of urine deliberately. In a further embodiment, the guiding device for urine collection further comprises a wing, which is extended from the body adjacent to the cup opening. The wing allows the user to be able to hold the wing by two fingers to operate the guiding device for urine collection so that the guiding device for urine collection is more closed to the body so that the guiding device for urine collection is more easily operation. In a further embodiment, the guiding device for urine collection further comprises a bag for collecting urine. The bag covers the first liquid discharge outlet or the second liquid discharge outlet. In some cases for convenient to carry, there is an opening provided on the bag for guiding the urine to a stool or a default destination.

### BRIEF DESCRIPTION OF THE DRAWINGS

The parts in the drawings are not necessarily drawn to scale, the emphasis instead being placed upon clearly illustrating the principles of at least one embodiment. In the drawings, like reference numerals designate corresponding parts throughout the various diagrams, and all the diagrams are schematic.

FIG. 1 is a schematic diagram showing one embodiment of the invention.

FIG. 2 is a schematic diagram showing another embodiment of the invention.

FIGS. 3A to 3D are schematic diagrams showing various shape of the overlooking profile of a cup opening.

FIG. 4 is a schematic diagram showing the cup opening with two wings of FIG. 2.

FIG. 5 is a schematic diagram showing the curved shape of the cup opening of FIG. 2.

FIGS. 6A to 6D are schematic diagrams showing the shape of the cup opening near the finger of FIG. 4.

FIGS. 7A and 7B are schematic diagrams showing the different position of a second liquid discharge outlet of FIG. 2.

FIG. 8 is a schematic diagram showing an guiding device for urine collection with a bag of FIG. 2.

### DETAILED DESCRIPTION

Reference will now be made to the drawings to describe various inventive embodiments of the present disclosure in detail, wherein like numerals refer to like elements throughout.

The present invention relates to a guiding device for urine collection is applied to the urine collection or to guide the urine to a specific destination. The guiding device for urine collection may be made of plastic, rubber, metal, paper, waterproof paper.

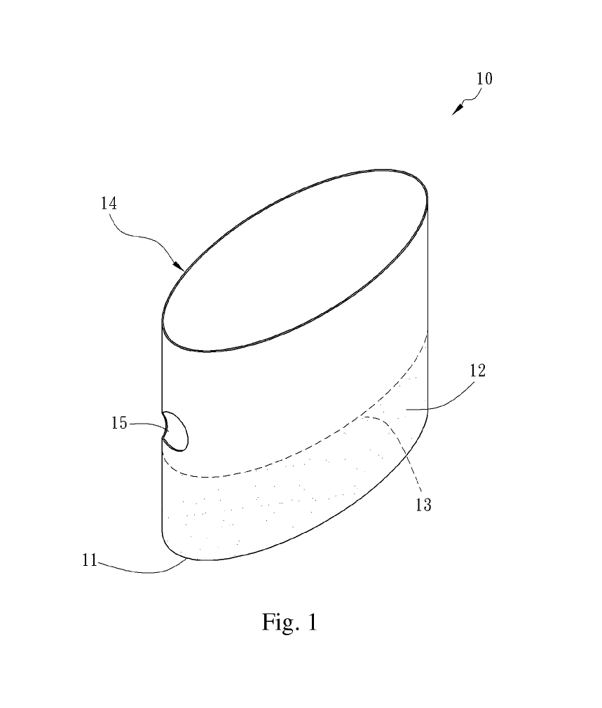

Refer to FIG. 1, a guiding device for urine collection 10 according to a first embodiment of the present invention includes a bottom portion 11, a body 12, a collection portion 13, a cup opening 14, and a first liquid discharge outlet 15. The body 12 is surrounded the bottom portion 11, and extending at an angle. The angle may be a right angle, an acute angle, or an obtuse angle. The collection portion 13 is formed by the bottom portion 11 and a part of the body 12 to collect or store the urine. The cup opening 14 is located at one side of the body 12, which is opposite to the bottom portion 11, to close to the body to cover the genitals of the female user. The first liquid discharge outlet 15 is disposed on the body 12, which is between the cup opening 14 and the collection portion 13.

The first liquid discharge outlet 15 is used to drain urine excess beyond the collection portion 13, then guide the urine temporarily stored in the collection portion 13 into the test tube (not shown in figure). Thus, the user can easily collect the urine. In addition, the first liquid discharge outlet 15 may be connected to a test tube (not shown in figure). The user can urinate when the cup opening 14 covering the genitals, and the urine is temporarily stored in the collection portion 13, and then tilt the guiding device for urine collection 10 to pour the urine into the test tube through the first liquid discharge outlet 15. In another embodiment, the urine also can be discharged into the test tube, which is connected to the first liquid discharge outlet 15.

As mentioned above, the guiding device for urine collection 10 of the invention utilizes the collection portion 13 to make users not easy to dirty the hands or clothing during the collecting process, and the urine stored in the collection portion 13 can be poured into the test tube by the first liquid discharge outlet 15. In other words, the level of the first liquid discharge outlet 15 is located above the level of the collection portion 13, so that the urine discharged into the test tube directly after collection process or during the collection process.

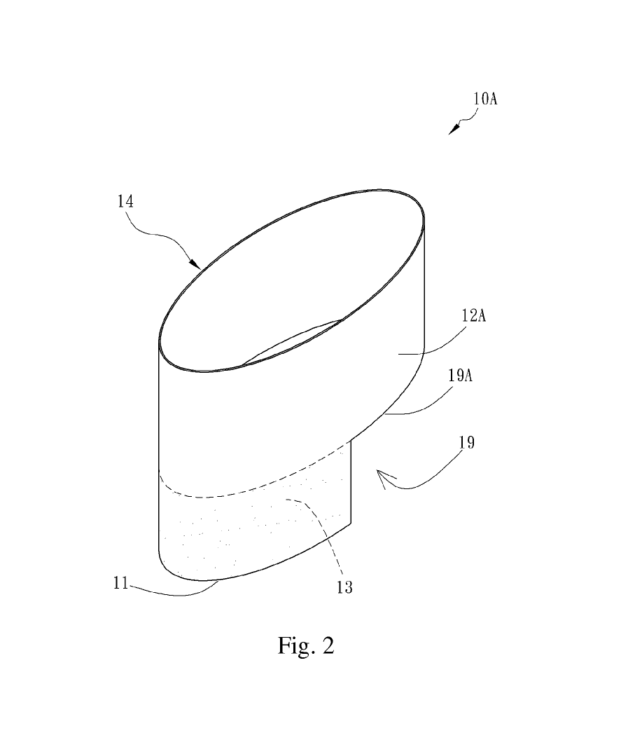

The guiding device for urine collection 10A shown in FIG. 2, the overall appearance of its body 12A has a concave portion 19 extended to the bottom portion 11. In operation status, the level of the first liquid discharge outlet 15 is higher than the collection portion 13. In the embodiment, the concave portion 19 has a top edge 19A. The first liquid discharge outlet 15 is located on the top edge 19A, and the collection portion 13 is located between the top edge 19A of the concave portion 19 and the bottom portion 11. In the embodiment, the top edge 19A and the bottom portion 11 is parallel to each other. In another embodiment, the top edge 19A or the bottom portion 11 can be a bevel, or can be a curved surface same as the side profile of the cup opening 14.

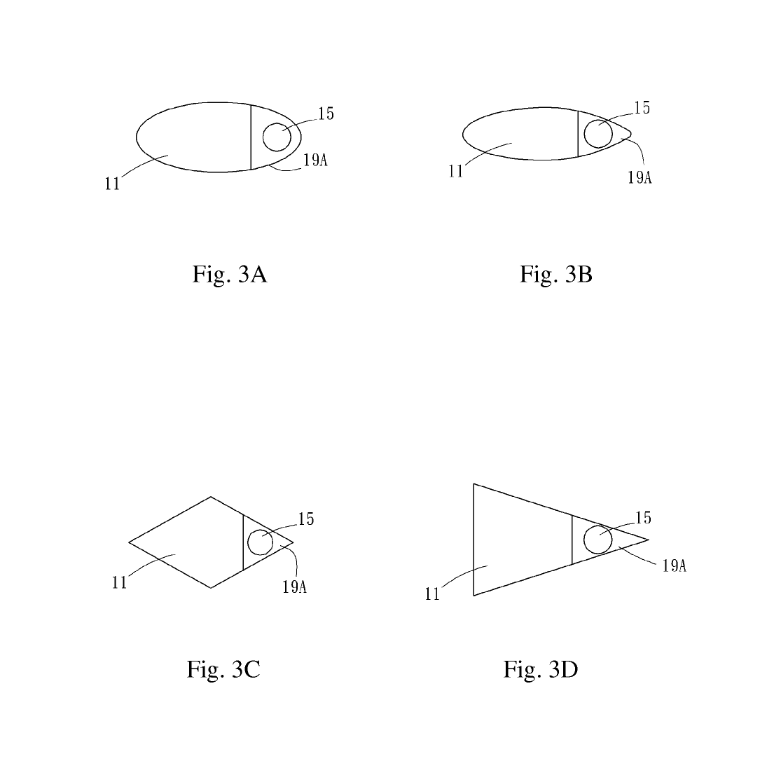

Referring to FIGS. 3A to 3D, an overlooking profile of the cup opening 14 may be circular, oval, teardrop-shaped, egg-shaped, rectangular, rhombic, or triangular, that is not to be limited.

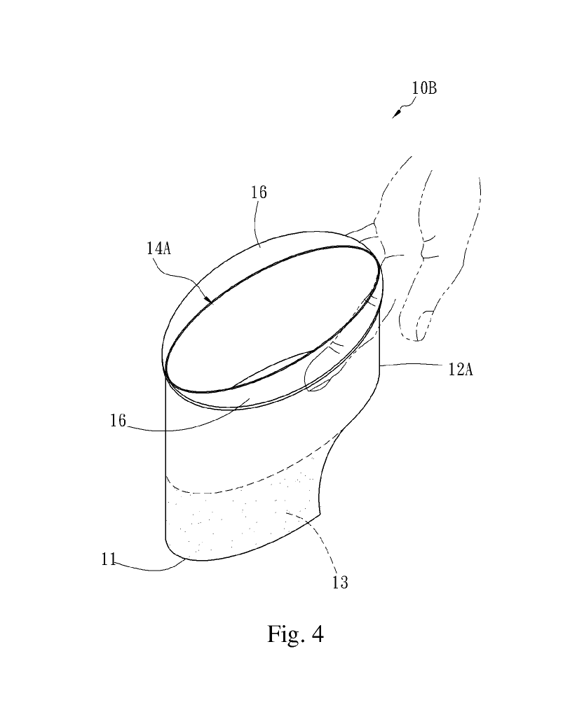

Referring to FIG. 4 and comparing to the FIG. 2, the guiding device for urine collection 10B according to a second embodiment of the present invention is further comprises at least one wing 16. The wing 16 is located at the body 12A, which is adjacent to the cup opening 14A to enable the user to use two fingers to stick the wing 16 and to operate the guiding device for urine collection 10B. An adhesive layer may be disposed on the wing 16 near the side of the finger, that is, the side of the wing 16 is distant from the cup opening 14A, so that the finger with any size can easily stick to operate the guiding device for urine collection 10B.

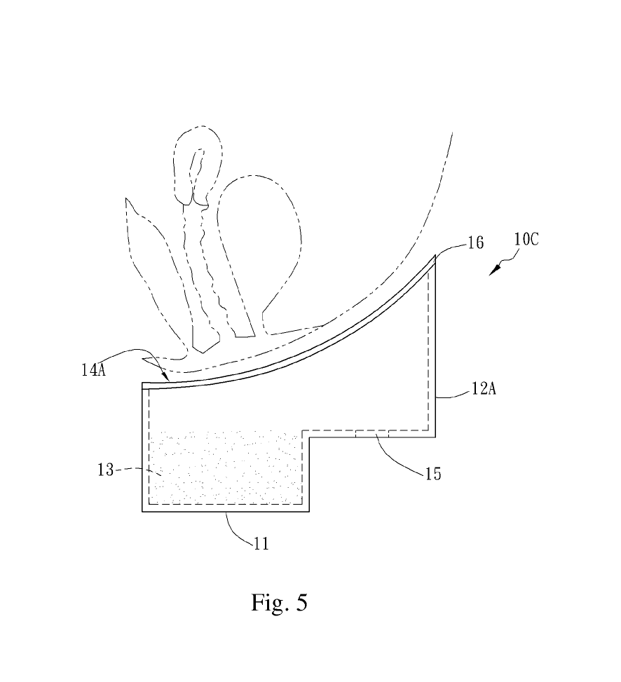

Referring to FIG. 5, the different between a guiding device for urine collection 10C according to a third embodiment of the present invention with the second embodiment described above is a side profile of the cup opening 14A that is a curved surface. The curved surface fits to the body curve of the genitals so that the cup opening 14A may more closely fit to the body to avoid urine overflow during urination.

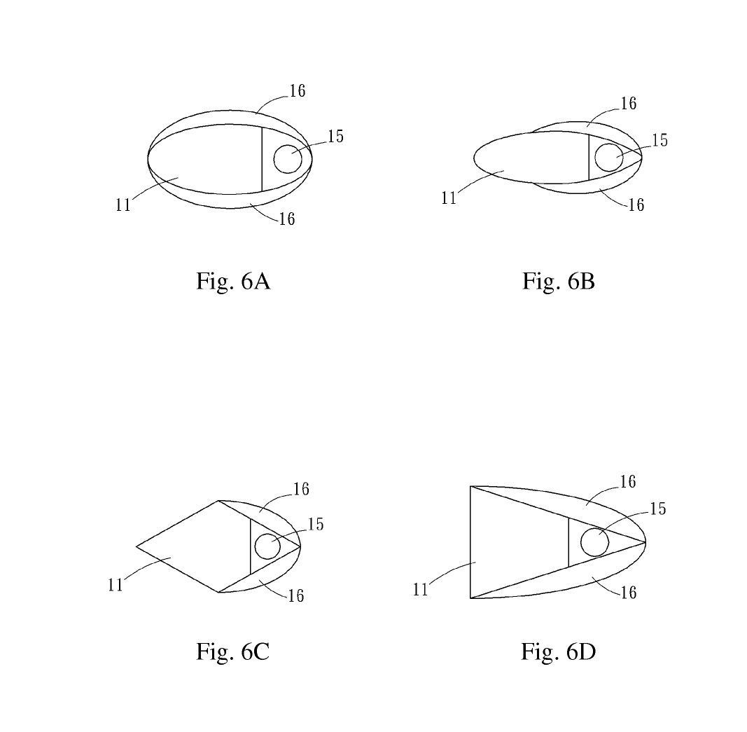

Due to the two fingers have a certain shape and angle when open the fingers , in the embodiment, the overlooking profile of the cup opening 14A close to the two fingers is sharp curved or v-shaped (shown as FIGS. 6A to 6D).

The first liquid discharge outlet 15 may be connected to a catheter to drain the urine to the default destination, for example, the stool. The first liquid discharge outlet 15 can also be connected to a test tube so that the urine can be directly poured into the test tube to avoid splashing of urine on hands, clothing, or the environment.

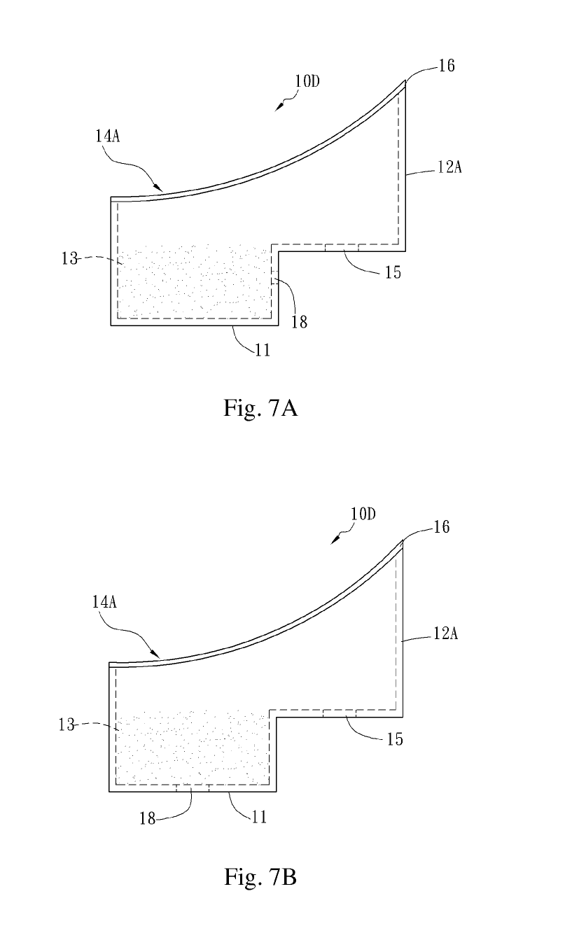

Referring to FIGS. 7A and 7B, a guiding device for urine collection 10D according to a fourth embodiment of the invention further comprises a second liquid discharge outlet 18, which may be located on the body 12A of the collection portion 13 (shown as FIG. 7A), or may be located on the bottom portion 11 (FIG. 7B). Similar to the first liquid discharge outlet 15, the second liquid discharge outlet 18 may be also connected to a catheter or a test tube. In the embodiment, for the urine collection, the second liquid discharge outlet 18 can be connected to the test tube, the first liquid discharge outlet 15 can be connected to the catheter, and one end of the catheter can be placed on a stool. In using the guiding device for urine collection 10D, which is closed to the body so that the cup opening 14A can cover the genitals. When urinating, the urine will enter the collection portion 13, and outflow to the test tube through the second liquid discharge outlet 18. The urine can be flowed from the catheter through the first liquid discharge outlet 15 when the urine is full of the test tube and the collected 13 and beyond the collection portion 13. Thus, the user do not need to the splashing of urine, and do not need to control the flow of urine by physiological mechanism so as to avoid the urine overflow from the cup.

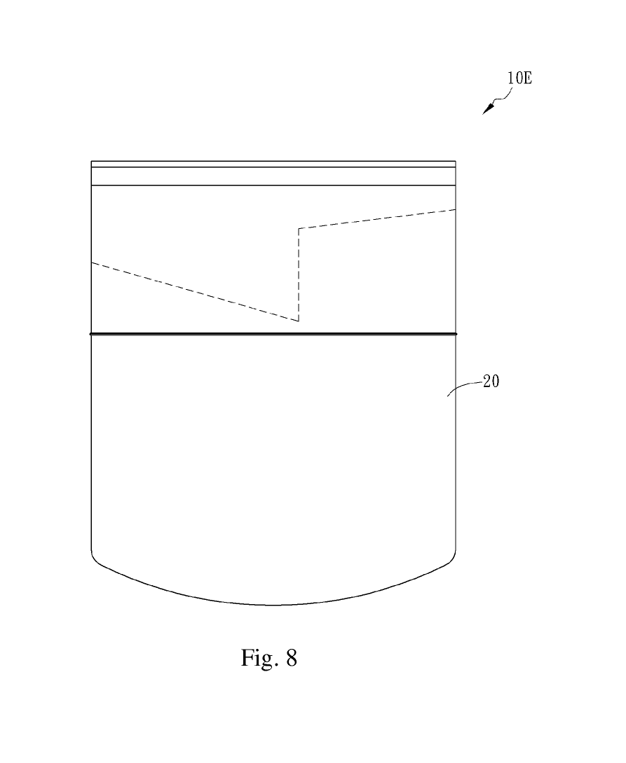

The bottom portion 11 of the guiding device for urine collection 10D may be an inclined surface. The inclined direction of the inclined surface is toward the direction of the first liquid discharge outlet 15 or the second liquid discharge outlet 18, which may guide the urine to flow to the first liquid discharge outlet 15 or the second liquid discharge outlet 18. Referring to FIG. 8, a guiding device for urine collection 10E according to the fifth embodiment of the invention further comprises a bag 20, which at least covers the first liquid discharge outlet 15, and may be connected to the body 12A by sleeved, stick, thermocompression bonding, or high frequency lamination. In the embodiment, the bag 20 is a zipper bag, which completely covers the first liquid discharge outlet 15 and the second liquid discharge outlet 18. The urine can be sealed in the zipper bag to prevent the urine leakage when there are no toilet nearby. In another embodiment, the bag 20 may only covering the second liquid discharge outlet 18, and the first liquid discharge outlet 15 is connected to the catheter or the test tube.

As mentioned above, the size or shape of the first liquid discharge outlet and the second liquid discharge outlet are not limited, especially the second liquid discharge outlet can even be the same or smaller than the size of the bottom portion. The guiding device for urine collection with the above description, the user will not need to deliberately control the flow of the urine and the urine can be discharged into the default destination, while easy to do the urine collection, or do not want to touch the dirty potty in public toilets.

Even though numerous characteristics and advantages of certain inventive embodiments have been set out in the foregoing description, together with details of the structures and functions of the embodiments, the disclosure is illustrative only. Changes may be made in detail, especially in matters of arrangement of parts, within the principles of the present disclosure to the full extent indicated by the broad general meaning of the terms in which the appended claims are expressed.

What is claimed is:

1. A guiding device for urine collection, comprising:
a bottom portion;
a body arranged to extend around the bottom portion, the bottom portion and a part of the body are formed a collection portion;
a cup opening located on a side of the body opposite to the bottom portion; and
a first liquid discharge outlet arranged on the body between the cup opening and the collection portion.

2. The guiding device for urine collection of claim 1, wherein a profile of the cup opening is a curved surface.

3. The guiding device for urine collection of claim 1, further comprises at least one wing located on the body adjacent to the cup opening.

4. The guiding device for urine collection of claim 1, wherein the body has a concave portion extended to the bottom portion, and the collection portion located between the bottom portion and an extension line of the top edge of the concave portion.

5. The guiding device for urine collection of claim 4, wherein the first liquid discharge outlet is located at a top edge of the concave portion.

6. The guiding device for urine collection of claim 1, wherein the overlooking profile of the cup opening is circular, oval, teardrop-shaped, egg-shaped, rectangular, rhombic, or triangular.

7. The guiding device for urine collection of claim 1, wherein one end of the overlooking profile of the cup opening is a sharp surface or v-shaped.

8. The guiding device for urine collection of claim 1, further comprises a second liquid discharge outlet located at the body of the collection portion or the bottom portion.

9. The guiding device for urine collection of claim 1, further comprises a bag connected to the body, and at least covering the first liquid discharge outlet.

10. The guiding device for urine collection of claim 9, wherein the bag is a zipper bag.
